(19) United States
(12) Patent Application Publication
(10) Pub. No.: US 2008/0160003 A1
(43) Pub. Date: Jul. 3, 2008

(54) Fertility Enhancement Using Lipid Carriers and Bioactive Molecules

(75) Inventors: Patricia A. DeLeon, Bear, DE (US); Genevieve S. Griffiths, Wilmington, DE (US)

Correspondence Address: MCCARTER & ENGLISH, LLP;BASIL S. KRIKELIS, Renaissance Centre, 405 N. King Street, 8th Floor, WILMINGTON, DE, 19801, US

(73) Assignee: University of Delaware, Newark, DE (US)

(21) Appl. No.: 11/928,962

(22) Filed: Oct. 30, 2007

Related U.S. Application Data
(60) Provisional application No. 60/855,500, filed on Oct. 31, 2006.

(51) Int. Cl. A61K 38/43; A01N 1/02; A61K 35/54

(52) U.S. Cl. 424/94.1; 435/2; 424/582

(57) ABSTRACT

The invention relates to a composition and method for enhancing fertilization. Fertilization enhancement is achieved by effectively delivering bioactive molecules with a lipid anchor (GPI-linked proteins) to the surface of epididymal or ejaculated sperm. The process may be facilitated or promoted in the presence of Clusterin/ApoJ, a well-known lipid carrier. The acquisition of these molecules, such as Sperm Adhesion Molecule 1 (SPAM1) can significantly impact sperm maturation and function.

### CROSS REFERENCE TO RELATED APPLICATIONS

This application claims the benefit of U.S. Provisional Patent Application Ser. No. 60/855,500 filed Oct. 31, 2006, the contents of which are herein incorporated by reference.

### RELATED FEDERALLY SPONSORED RESEARCH

The work described in this application was sponsored by the National Institutes of Health (NIH) under Contract Number ROI HD38273.

### FIELD OF THE INVENTION

The invention relates to a composition and method for enhancing fertilization. Fertilization enhancement is achieved by effectively delivering bioactive molecules with a lipid anchor (GPI-linked proteins) to the surface of epididymal or ejaculated sperm. The process may be facilitated or promoted in the presence of Clusterin/ApoJ, a well-known lipid carrier. The acquisition of these molecules, such as Sperm Adhesion Molecule 1 (SPAM1), can significantly impact sperm maturation and function.

### BACKGROUND OF THE INVENTION

Cell-to-cell transfer of glycosyl phosphatidylinositol (GPI)-linked membrane proteins in vivo is known thus far for sperm and erythrocytes, cell types in which biosynthetic ability is absent or limited. This transfer plays a pivotal role in the remodeling of the sperm plasma membrane (PM) during their maturation in both the male and female genital tracts.

Although sperm leaving the testis are incapable of transcriptional and translational activity, their surface proteins undergo a remarkable degree of modification during epididymal maturation and capacitation in the female tract. During epididymal transit (which may vary from 3-12 days depending on the species) sperm are in an intimate association with the epididymal epithelium and its secretions and thereby exposed to variety of macromolecules that are sequentially added to their PM surface. After epididymal transit, however, sperm are not fully mature and ready to fertilize an egg. During capacitation in the female, molecules are added to sperm from the secretions of the female tract, where sperm reside for a shorter period. Some of these modifications on the sperm surface result from exchanges between soluble lipid donors or acceptors and the PM, and a variety of the proteins involved are GPI-linked. After capacitation in the female tract, sperm are fully mature and ready to fertilize an egg.

Sperm surface remodeling plays an important role in fertilization. The addition of bioactive molecules on the surface of sperm furthers post-testicular maturation. This remodeling increases the likelihood of successful fertilization with an egg. Deficiencies in sperm surface remodeling, likewise, lead to a reduction in egg fertilization. The inventors have discovered a new composition and method of enhancing fertilization by promoting the remodeling of the sperm surface.

### SUMMARY OF THE INVENTION

The present invention is directed to a composition comprising a substantially purified bioactive molecule, such as GPI-linked proteins, enzymes, adhesion molecules, immune proteins and glycoproteins, and a substantially purified lipid carrier. The composition may comprise a bioactive molecule and a substantially purified lipid carrier. GPI-linked proteins may be sperm adhesion molecule 1 (SPAM1) or P34H. The lipid carrier may be a lipid transport protein, such as Clusterin, ApoJ, Clusterin/ApoJ, ApoA-1, SGP2, TRPM, gp80 or SP-40. The bioactive molecule may be naturally occurring, synthetic or recombinantly derived.

The present invention is also directed to a method of enhancing fertilization comprising administering to an animal, male or female, a composition comprising a substantially purified bioactive molecule and a substantially purified lipid carrier. The composition may comprise a substantially purified bioactive molecule and a lipid carrier. The composition may comprise a bioactive molecule and a substantially purified lipid carrier. The method may be used to transfer the bioactive molecule from the composition to the surface of a sperm cell in the animal.

The present invention is also directed to an in vitro method for enhancing sperm maturation and function before or after IUI, the method comprising the steps of, isolating sperm from a male candidate and combining, in vitro, said sperm in a medium supplemented with at least one lipid carrier and/or said GPI-linked molecule and incubating for a predetermined amount of time.

The present invention is also directed to a method for enhancing sperm maturation and function before in vitro fertilization, the method comprising, the steps of isolating sperm from a male candidate, in an in vitro environment, capacitating said sperm in a capacitation medium wherein the capacitation medium is supplemented with at least one lipid carrier or at least one protein and incubating for a predetermined amount of time.

The present invention is also directed to a method for delivery of a GPI-linked molecule, naturally or recombinantly derived, to sperm in intrauterine insemination (IUI) or in in vitro fertilization (IVF), the method comprising the step of combining, in vitro, said sperm in a medium supplemented with said GPI-linked molecule and incubating for a predetermined amount of time.

The invention is also directed to a method for in vitro fertilization, the method comprising the steps of (a) obtaining an egg from a female candidate; (b) isolating sperm from a male candidate; (c) capacitating, in vitro, said sperm in a capacitation medium supplemented with at least one lipid carrier and/or at least one GPI-linked protein; (d) fertilizing, in vitro, said egg with sperm to produce at least one fertilized egg; (e) culturing said fertilized egg to produce an embryo; and (f) transferring at least one embryo to the uterus of an animal.

The invention is also directed to a method for intrauterine insemination, the method comprising the steps of (a) isolating sperm from a male candidate; (b) combining, in vitro, said sperm in a medium supplemented with at least one lipid carrier and/or at least one GPI-linked protein; and (c) thereafter, introducing said sperm into the uterine tract of an animal wherein the sperm fertilize an egg.

The invention is finally directed to a method for removing a GPI-linked protein from a cell that recombinantly expresses the GPI-linked protein, the method comprising the step of adding at least 40 ug/mL of a lipid carrier to said cell prior to removal.

The GPI-linked proteins may be selected from the group consisting of sperm adhesion molecule 1 (SPAM1) and P34H. The lipid carrier may be selected from the group consisting of Clusterin, ApoJ, Clusterin/ApoJ, ApoA-1, SGP2, TRPM, gp80 and SP-40.

### BRIEF DESCRIPTION OF THE DRAWINGS

The file of this patent contains at least one drawing executed in color. Copies of this patent with color drawings(s) will be provided by the Patent and Trademark Office upon request and payment of the necessary fee.

FIG. 1 is an illustration of a GPI-linked protein showing the acyl chain which anchors it in the external leaflet of the lipid bylayer. The C-terminal amino acid of the protein is linked to the inositol phospholipids anchor through a core glycan structure.

FIG. 2 is a model showing lipid exchange at the surface of sperm within the epididymis and uterus.

FIG. 3 shows an association of SPAM1 and ApoJ in co-immunoprecipitation (IP) from ELF and ULF. The presence (+) of ApoJ and SPAM1 Ab, control preimmune serum (PIS), and the Ab used for Western blots (WB) are indicated. In (A) the ˜67 kDa SPAM1 band is seen for ELF and ULF and is precipitated by ApoJ Ab. In (B), where the proteins are unreduced, an ˜70 kDa ApoJ band is precipitated by SPAM1 Ab in both ULF and ELF (arrow).

FIG. 4 shows a comparison of SPAM1 uptake. SPAM1 uptake is hindered by increasing lipoprotein concentrations.

FIG. 5 shows the effect of lipoprotein concentration in ULF on SPAM1 uptake. Low concentrations of lipoprotein supplements in ULF enhance the uptake of SPAM1 measured by flow cytometric analysis.

FIG. 6 shows the effect of lipoprotein on SPAM1 uptake for human and mouse sperm. SPAM1 is removed from mouse sperm (A) after treatment with exogenous lipoproteins. The control (grey) was PBS-treated. B shows human sperm incubated in varying concentrations of lipoproteins with solubilized human sperm PM proteins. While transfer occurred in all samples, the efficiency was highest at 16 μg/mL. In A and B, 50,000 cells were analyzed for each sample by flow cytometry.

FIG. 7 shows HASGE analysis of sperm protein. HASGE analysis of 20 μg sperm protein loaded in each lane. Lane 1 has mouse proteins. Lanes 2-7 are human samples. Lane 4 has no hyaluronidase activity. Compared to 3, Lanes 2, 5-7 have varying degrees of reduced activity.

### DETAILED DESCRIPTION OF THE INVENTION

The objective of this invention is to enhance fertilization in animals.

Another objective of this invention is to supply the sperm surface with biologically or biomedically-relevant membrane-free molecules that will enhance the sperm's functional ability.

Another objective of this invention is to enhance the ability of sperm to effect fertilization in vitro, as well as after intrauterine insemination.

As used herein, the term “substantially purified” refers to naturally occurring, synthetic or recombinant compounds that are at least 80% pure. Preferably, the compounds are at least 85% pure. More preferably, the compounds are at least 90% pure. Even more preferably, the compounds are at least 95% pure. And even more preferably, the compounds are at least 99% pure. And even still more preferably, the compounds are at least 99.9% pure.

As used herein, the term “bioactive molecule” refers to a molecule that can be present or found in epididymal and/or uterine secretion. Some examples of such molecules include GPI-linked proteins, enzymes, adhesion molecules, immune proteins, antigens and glycoproteins. These bioactive molecules may be naturally occurring, synthetic or recombinantly derived. Bioactive molecules may also be referred to as “surface remodeling,” such as “surface modeling proteins.” Bioactive molecules of the invention are preferably membrane-free and have biological and/or biomedical relevance to a sperm's functional characteristics.

As used herein, the term “GPI-linked protein” refers to proteins that can attach to the surface of the sperm by glycosy phosphatidylinositol linkage, such as SPAM1, P34H, CD52, CD55, CD59, and CD73.

As used herein, the term “lipid transport protein” or “lipid carrier” refers to a compound that transports bioactive molecules to and from the sperm surface, such as Clusterin, ApoJ, Clusterin/ApoJ, ApoA-1, SGP2, TRPM, gp80 and SP-40.

As used herein, the terms “intrauterine insemination” and “in vitro fertilization” refer to such assisted reproduction methods known in the art and include intrauterine insemination (IUI), intracervical insemination, embryo transfer and gamete intrafallopian transfer. Such methods are useful for assisting males and females who may have physiological or metabolic disorders that prevent natural conception. They may be used to enable females to bear progeny who are otherwise unable to conceive naturally. In addition to use in humans, such methods are also useful in animal breeding programs, such as for livestock breeding, and could be used as methods for the creation of transgenic animals. Bioactive molecules of this invention can be combined with sperm, an egg or an egg-sperm mixture prior to fertilization of the egg. In some species, sperm capacitate under in vitro conditions spontaneously during in vitro fertilization procedures, but normally sperm capacitate over an extended period of time both in vivo and in vitro. It is advantageous to enhance sperm activation during such procedures to increase the likelihood of successful fertilization.

As used herein, the term “capacitation” and “capacitate” refer to the specific changes a sperm undergoes in the uterine tract to develop the capacity to fertilize ova, such as protein changes on the surface or associated with the plasma membrane facilitate penetration of the sperm into the ovum. Sperm maturation occurs over a continuum, which is described as three stages. The first stage of sperm maturation occurs in the testis where sperm is generated. Sperm present in the testis are immature and not ready to fertilize an egg. The second stage is epididymal maturation which occurs in the male tract. After epididymal maturation sperm are not fully mature and are not ready to fertilize an egg. The final stage of maturation is capacitation which occurs in the female tract. After capacitation, sperm are ready to fertilize an egg.

Prior to fertilization after natural mating epididymally mature sperm undergo a final maturation period, capacitation, in the female tract during which they are prepared for interaction with the eggs. Since ejaculated sperm are unable to fertilize eggs immediately on contact with eggs in vitro, capacitation is often considered an essential pre-requisite for the fertilization process. Thus for in vitro fertilization the process is simulated prior to the introduction of the sperm to the egg.

As used herein, the term “capacitation medium” or “capacitating medium” refers to a solution that facilitates capacitation of sperm. A capacitating medium may include a variety of ingredients such as calcium, sodium lactate, sodium pyruvate, HEPES buffer, and sodium bicarbonate and bovine serum albumin among others. An effective capacitation medium for the invention is Human Tubal Fluid (HTS) which is commercially available from sources such as Millipore (EMBRYOMAX® Human Tubal Fluid). In addition the capacitation medium may contain uterine fluid, epididymal fluid, human tubal fluid or synthetic uterine fluid which facilitates capacitation of sperm. Any applicable capacitation medium known to those of skill in the art may be used. As used herein, the term “medium” refers to a solution that facilitates the combining of sperm and either bioactive molecules or lipid carriers. Any applicable medium known to those of skill in the art may be used.

Further, in vitro capacitation is known to occur under certain specified conditions which include a sterile environment, capacitating medium, 37° C., and an atmosphere of reduced O₂. The period of sperm capacitation varies with the species. For example, in the mouse, in vitro capacitation generally takes 45 to 60 minutes in the above conditions.

In example 3, we conveniently combine uptake of epididymal proteins, including SPAM 1, with in vitro capacitation of epididymally mature caudal sperm to enhance the fertilizing capacity of sperm.

Cell-to-cell transfer of glycosyl phosphatidylinositol (GPI)-linked membrane proteins in vivo is known thus far for sperm and erythrocytes, cell types in which biosynthetic ability is absent or limited. This transfer plays a pivotal role in the remodeling of the sperm plasma membrane (PM) during the sperm's maturation in both the male and female genital tracts.

Although sperm leaving the testis are incapable of transcriptional and translational activity, their surface proteins undergo a remarkable degree of modification during epididymal maturation and capacitation in the female tract. During epididymal transit (which may vary from 3-12 days depending on the species) sperm are in an intimate association with the epididymal epithelium and its secretions and thereby exposed to variety of macromolecules that are sequentially added to their PM surface. After epididymal transit, however, sperm are not fully mature and ready to fertilize an egg. In the female, molecules are added to sperm from the secretions of the female tract, where sperm reside for a shorter period. Some of these modifications on the sperm surface result from exchanges between soluble lipid donors or acceptors and the PM, and a variety of the proteins involved are GPI-linked. After capacitation in the female tract, sperm are fully mature and ready to fertilize an egg.

Sperm surface remodeling plays an important role in fertilization. The addition of bioactive molecules on the surface of sperm furthers post-testicular maturation. This remodeling increases the likelihood of successful fertilization with an egg. Deficiencies in sperm surface remodeling, likewise, lead to a reduction in egg fertilization. The inventors have discovered a new composition and method of enhancing fertilization by promoting the remodeling of the sperm surface and thus empowering the sperm to fertilize.

Bioactive molecules of the invention that enhance fertilization by attachment to the sperm surface include, for example, GPI-linked proteins, enzymes, adhesion molecules, immune proteins, antigens and glycoproteins.

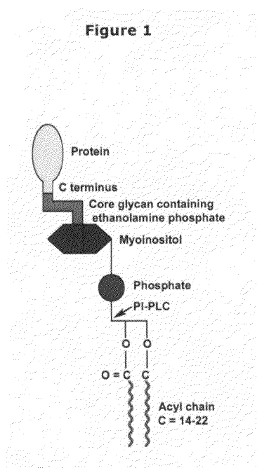

GPI-linked proteins include membrane-associated enzymes and adhesion molecules, among a variety of other glycoproteins. They are anchored to PMs post-translationally via a covalent attachment of glycosylated phosphatidylinositol molecules (FIG. 1) and are confined to the outer leaflet of the lipid bilayer, usually in microdomains which are rich in glycosphingolipids and cholesterol. Some GPI-linked proteins are associated with exosomes or vesicles called epididymosomes which are characterized by a high cholesterol/phospholipid ratio, and many are associated with germ cells. Others are released by apocrine secretion resulting from blebbing of the epithelial lining. Preferred examples of GPI-linked proteins include SPAM1, P34H, CD52, CD55, CD59, and CD73.

As seen in FIG. 1, after triggering the acrosome reaction and the secondary binding of sperm to the zona pellucida, early steps in fertilization, GPI-linked proteins are cleaved in the glycan core by angiotensin-converting enzyme (ACE), an endomannosidase, from the sperm tail's midpiece. This cleavage facilitates further sperm-egg interaction by functional activation of the proteins or removal of the physical barrier they represent to sperm-egg interaction (Kondoh et al., 2005). The addition of surface proteins to the sperm surface by an attachment other than the GPI linkage results in only limited functional activity and maybe counterproductive if it is not able to be cleaved by ACE.

A large number of GPI-linked proteins are involved in reproduction. GPI-linked proteins that were initially shown to be acquired by post-testicular sperm in vivo were ones that were also found on cells in the immune system (e.g. CD52, CD55, CD59, CD73); thus they were thought to be involved solely in protecting sperm from immune attack in the male and female tract. However, it has now become clear that the distribution/translocation of GPI-linked proteins on the sperm PM during post-testicular maturation underscores the importance of this type of PM attachment directly in the mammalian reproductive process. Facilitated by their unhindered lateral mobility, these proteins are known to participate in epididymal maturation, the signal transduction process in capacitation, acrosomal exocytosis, and sperm-egg interaction. Compared to other types of PM attachments for sperm proteins, the GPI-anchor offers special structural and functional advantages. It facilitates lateral diffusion which not only economizes on the number of required molecules, but improves the dispersion and interaction with other molecules on the sperm PM.

Recently, three fertility centers in Canada showed that a lack of protein P34H, known to be involved in sperm-egg interactions, can be used as a predictor of cases of failed fertilization treatments. (Moskovtsev S. I., et al. Epididymal P34H protein deficiency in men evaluated for infertility. Fertil Steril. 2007 Apr. 13. and Boue F., et al. Cases of human infertility are associated with the absence of P34H an epididymal sperm antigen. Biol Reprod 54:1018-1024, 1996). P34H is another GPI-linked protein that may be used within the present invention to enhance sperm.

During sperm surface remodeling, there is the loss of surface proteins and the selective absorption of epididymal, uterine and oviductal factors on the PM. In the female tract, cholesterol efflux from the sperm PM is known to play an important role in capacitation. Although the mechanism of the efflux is not well understood, there is convincing evidence for the involvement of high density lipoprotein (HDL) and other lipid complexes which serve as acceptors of sperm cholesterol and phospholipids. ApoJ/Clusterin and ApoA-1 are implicated in the process of lipid exchange from the sperm PM to epithelial cells of the epididymis and uterus.

Clusterin is a family of multifunctional secretory glycoprotein that is expressed is a variety of body fluids. Some examples of clusterin glycoproteins include ApoJ, SGP2, TRPM, gp80 and SP-40. It is known as a chaperone-like protein that can bind lipids and membrane-active proteins and is abundantly expressed in testis (specifically Sertoli cells), epididymis and in the female genital tract, although its specific function has long been the subject of much speculation. Importantly, it is expressed on the surface of sperm and due to its abundance and spatial expression pattern is thought to play an important role in sperm development and maturation. A major fraction of ApoJ in the ELF is free or loosely associated with sperm while a smaller fraction is tightly associated with the lipid bilayer. Further, epididymal ApoJ forms complexes with other proteins and or/lipids, but not specifically ApoA-1. More recently, it has been shown to be involved in lipid exchange in the male tract where the lipidated protein is endocytosed via a receptor-mediated mechanism at the epithelial cell lining. Expression of ApoJ and its receptor, Megalin (LRP2), in the male parallels that in the female where the receptor is present in the uterine and oviductal epithelia. It is also maximally expressed during estrous and metestrous.

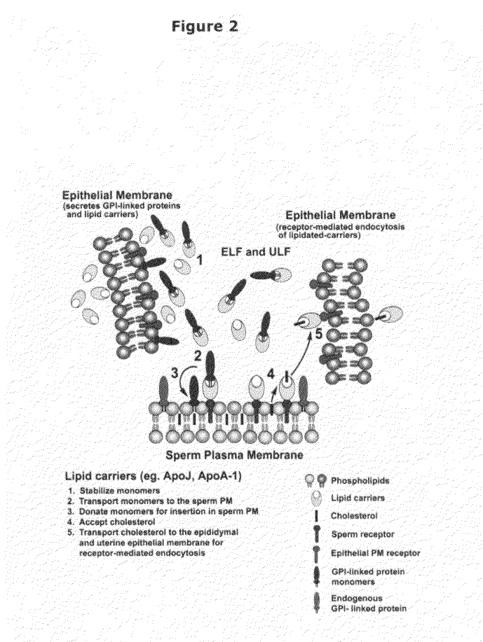

Apolipoprotein A-1 (ApoA-1) is a major protein of plasma HDL and is known to play important roles in lipid transport and metabolism. It has also been shown to bind to a family of bovine seminal plasma proteins. Like ApoJ, it is also expressed in the male and female where it is implicated in the process of lipid exchange from the sperm PM to that of the epithelial cells. It shares with ApoJ the same receptor (Megalin) and along with a co-receptor, Cubulin, it mediates endocytotic removal of lipidated proteins. While ApoJ has been demonstrated to bind to the sperm surface, this has not been clearly shown for ApoA-1. FIG. 2 is a model showing lipid exchange at the surface of sperm within the epididymis and uterus.

Mammalian epididymal luminal fluid (ELF) has been shown to be a complex consisting of particulate membranous vesicles and soluble membrane-free components. This has also been shown to be characteristic of uterine luminal fluid (ULF). However, capacitation takes place in ULF or simulated ULF. Simulated ULF may contain ELF. Importantly, Sperm adhesion molecule 1 (SPAM1), among a number of other GPI-linked proteins present in mouse ELF and ULF, can be acquired on the sperm surface in vitro from both components, with uptake being more efficient from the soluble membrane-free fraction. Sub-fractionation of this soluble component by ultracentrifugation (230,000×g) revealed the presence of oligomeric aggregates in the pellet and predominantly soluble SPAM1 monomers (67 kDa). It has also been shown that SPAM1 uptake from this sub-fraction is modulated by the presence of added exogenous lipoproteins: there was found to be an inverse relationship between the concentration of lipoproteins and SPAM1 transfer to the sperm surface.

SPAM1 is an ideal model for elucidating the mechanisms of sperm uptake and removal of GPI-linked proteins. The specific function(s) of most of the GPI-linked proteins acquired by sperm are unknown. However SPAM1, which is the major mammalian sperm hyaluronidase, plays multifunctional roles in fertilization and is ideal for the studies proposed. Our lab has shown it to be a secretory protein in the epididymides of humans, macaques, rats and mice, and expression appears to be conserved. In mice it has been shown to be expressed in all three regions (the efferent ducts, epididymis, and vas deferens) of the male tract, as well as the accessory organs (prostate, and seminal vesicles). The secretions from all three regions (caput, corpus, cauda) of the mouse epididymis were shown to contain SPAM1 in both a soluble (120S) and vesicular form (120P) (40:60), with the latter having an intact GPI anchor. More recently it has been shown that when Spam1 null sperm are exposed in vitro to unfractionated ELF there was considerable acquisition of SPAM1 and this was accompanied by a significant increase in cumulus penetration. This suggests that epididymal SPAM1 plays a role in sperm PM remodeling and is a marker sperm maturation.

Our lab has shown that SPAM1 is also expressed in all three regions (vagina, uterus, oviduct) of the female genital tract cyclically. It is present predominantly during estrus and is located in both the glandular and the secretory epithelium. More recently, it has been shown that it is secreted in the ULF in both a soluble and a vesicular form, and is also present in the oviductal fluid. Importantly, in vitro SPAM1 uptake by Spam1 null sperm from unfractionated wild type (WT) ULF showed a localization that mimicked that of WT mature sperm, as was the case for uptake from ELF. It is interesting that SPAM1 is associated with lipid rafts which are rich in cholesterol and GPI-linked proteins. It should be noted that lipoproteins such as ApoJ could function efficiently in donating their stabilized GPI-linked proteins in the same location that they remove cholesterol.

Soluble lipid carriers, thought to play a role in cholesterol efflux from the sperm plasma membrane, are also responsible for stabilizing soluble GPI-linked monomers and facilitating their insertion via their acyl chains into the outer leaflet of the lipid bilayer. In vitro acquisition of SPAM1 on the surface of caudal mouse sperm from the membrane-free monomeric component of both ELF and ULF is dependent on the presence of Clusterin/ApoJ, a lipid carrier abundantly expressed in the genital tracts. When ApoJ in ELF and ULF was antibody-inhibited in the soluble monomeric sub-fraction, SPAM1 uptake on mouse sperm was markedly reduced.

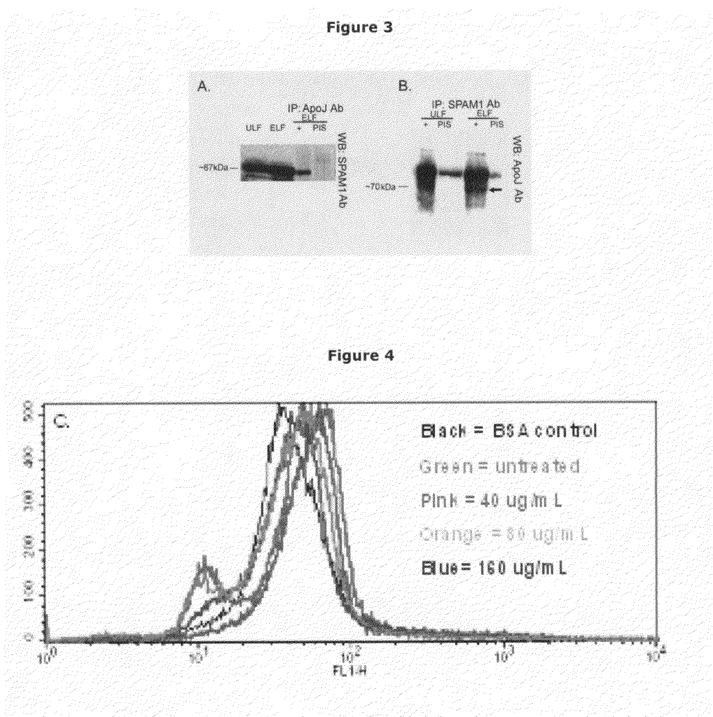

In addition, we have shown an association of SPAM1 and ApoJ in immunoprecipitations from the luminal fluids, reflecting the intimate interaction of these proteins. ApoJ is known to bind to the sperm surface. In FIG. 3 is shown a Western blot that indicates reciprocal co-immunoprecipitation of SPAM1 and ApoJ. This finding reveals that the proteins have an association which is likely mediated by hydrophobic interactions. Such interactions identify a role for ApoJ in the transfer of SPAM1 and other GPI-linked proteins from LFs to the sperm plasma membrane. This is the first identified interaction between SPAM1 and ApoJ. Interestingly, epididymal soluble prion protein which is GPI-linked was recently shown to form complexes with ApoJ. (Ecroyd, H., et al. The epididymal soluble prion protein forms a high-molecular-mass complex in association with hydrophobic proteins. Biochem J 392: 211-219, 2005).

ApoJ in ELF and ULF stabilizes monomers of GPI-linked proteins, transports them to the sperm surface where they are inserted into the plasma membrane during epididymal maturation and capacitation. This model extends the currently held view that during cholesterol efflux at the sperm membrane lipid-poor ApoJ accepts cholesterol and transports it the epididymal and uterine epithelial membranes for receptor-mediated endocytosis. Our work shows a novel role for ApoJ whose exact function has been an enigma for some time. It also has the potential of leading to advances in technology for the delivery of biologically or biomed ically relevant membrane-free GPI-linked molecules to the sperm surface before IUI or IVF, to enhance sperm maturation and function. The present invention also extends beyond the reproductive field.

The advantage is that the acquisition of these proteins occurs from membrane-free molecules rather than membranous vesicles. These membrane-free molecules, as well as Clusterin, can be made recombinantly, and used for in vitro interaction with the sperm surface.

This invention deals with an understanding of the physical and chemical interactions that determine the precise delivery of GPI-linked molecules in vitro to the sperm plasma membrane. We have determined that delivery is most efficient from monomers compared to vesicles or oligomeric aggregates, and that delivery of these monomers is enhanced in the presence of at least one lipid carrier, Clusterin or ApoJ. ApoJ has long been known to be present in abundant quantities in the male and female tracts and to be a chaperone molecule. Its precise function has not been clearly delineated, although it is thought to help to bring about the net efflux of cholesterol that occurs at the sperm surface during their maturation in the male and female environments. It is thought to act as an acceptor of cholesterol which is then disposed of at the epithelial membrane lining the epididymal and uterine tract by a process of receptor-mediated exocytosis.

We have found that when Clusterin binds to the sperm membrane it also acts as a donor of lipid molecules to the sperm surface. Using the SPAM1 model, we have shown that antibody blockage of ApoJ in the luminal fluid from both the male and female tract considerably inhibits the uptake of this protein. We have also shown that when various amounts of exogenous lipoproteins were added to the soluble fraction of the epididymal luminal fluid there was an inverse relationship between concentration and SPAM1 transfer to the sperm plasma membrane, implicating the involvement of lipoproteins in general in the delivery of GPI-linked proteins.

To confirm the involvement of ApoJ in the transfer of GPI-linked proteins to the sperm surface, we used immunoprecipitation to show an intimate association between SPAM1 and ApoJ and vice versa (See Example 5). This is the first reported interaction between clusterin/ApoJ and SPAM1 in both the epididymal luminal fluid (ELF) and the uterine luminal fluid (ULF). Based on the large number of GPI-linked proteins involved in reproduction, Clusterin is likely to play an important role in the uptake of proteins from the liquid phase of the luminal fluids.

Alternative uses of the invention included, but are not limited to, a method for GPI-transfer technology to express on the cell surface biologically important molecules, might be useful in a variety of ways, e.g. anticancer and antiviral immunotherapy. There are also implications that include disease transmission with respect to prions which have GPI anchors and are known to be added to the sperm surface at ejaculation in rams.

From a theoretical or fundamental point of view, it significantly increases the understanding of the coupled processes of epididymal sperm maturation and capacitation, with respect to the acquisition of GPI-linked proteins in the remodeling of the sperm PM. We have found a novel lipid donor and stabilizer role for the well-known lipid acceptors, ApoJ and ApoA-1, known to be involved in sperm maturation, in that they could stabilize monomers of SPAM1 and other GPI-linked proteins in the LFs, and deposit them at the sperm PM for insertion prior to removing cholesterol. Thus, the invention increases the understanding of sperm surface lipid exchange involving the net efflux that occurs during sperm maturation. Specifically, it reveals a more efficient interaction of lipid acceptors and the sperm PM than previously envisaged in cholesterol efflux.

The invention provides a means of adding bioactive molecules, such as SPAM1, P34H or other GPI-linked proteins, to the surface of sperm during the processing that precludes both intrauterine insemination (IUI) and in vitro fertilization (IVF). Prior to the present invention, the only recombinant source of SPAM1 available was a recombinant SPAM1 without the GPI-link or anchor. Human recombinant SPAM1 without the GPI-link/anchor was shown to be 10× more effective than slaughterhouse-derived SPAM1 in the dissolution of the cumulus cells, when mixed with sperm in IVF. (Bookbinder, L. H., et al. A recombinant human enzyme for enhanced interstitial transport of therapeutics. J. Control Release 114: 230-241, 2906 and Kunda, A., et al. Dispersion of cumulus matrix with a highly purified recombinant human hyaluronidase (rHuPH20). Hyaluronan 2003, The Cleveland clinic and Matrix Biology Institute, Poster Session #8, October 11-16, Cleveland, Ohio.). SPAM1 without the GPI-link could be produced recombinantly because it was removable (i.e. solubilized) from the cell by known techniques. Conversely, recombinant synthesis of SPAM1 with the GPI-link intact cannot be removed or solubilized from the cell by known techniques. The GPI-link, however, is necessary for SPAM1 to attach to sperm. The development of a recombinant SPAM1 with anchor intact would be significantly advantageous as sperm acquisition of such a recombinant GPI-linked SPAM1, in addition to increasing cumulus penetration, enhances the signaling involved in acrosomal exocytosis and zona binding, functions unattainable with the current soluble recombinant protein having no lipid anchor.

The present invention provides the technology of obtaining such a superior human recombinant SPAM1 with an intact GPI anchor for use in IUI and IVF. We have discovered that clusterin, when added to epididymal proteins at high levels, inhibits the uptake of SPAM1. At high levels, clusterin can remove SPAM1 and other GPI anchored proteins from the cell surface. High levels are considered to be about at least 40 ug/mL. Preferred high levels of lipid carrier are about 40 to about 2,000 ug/mL. More preferably, high levels of lipid carrier are about 100 to about 1,000 ug/mL. Effective removal of GPI-linked protein was performed using 800 ug/mL of clusterin. In addition, FIG. 4 shows that SPAM1 uptake is hindered by increasing the lipid carrier concentration.

Similarly, the invention may be used to supply bioactive molecules to patients in whom a lack of bioactive molecule on the sperm surface is detected. Based on the large number of GPI-linked proteins involved in reproduction the present invention is expected to have a far-reaching impact on the reproductive field.

The method of delivery of these bioactive molecules, such as P34H and SPAM1, to the sperm surface is non-invasive. In IUI, recombinant bioactive molecule, such as SPAM1 or P34H, along with recombinant carrier, such as ApoJ can be added to the insemination media, such as up to 60 minutes while in the catheter bag, prior to insemination. In the case of IVF fresh or frozen sperm that have undergone purification, such as by Pure Sperm Separation, can be treated with recombinant GPI-linked proteins along with a carrier, such as ApoJ, prior to being placed in human tubal fluid and before the final wash after which they are placed in culture medium for inseminating oocytes.

FIG. 5 shows that low concentrations of lipoprotein supplements in uterine luminal fluid (ULF) enhance the uptake of SPAM1 in flow cytometric analysis. Sperm uptake of SPAM1 from the soluble ULF fraction was dramatically enhanced when rat serum lipoproteins (mixed with preimmune serum (PIS) (1:100)) were added at a final concentration of 5-20 μg/mL prior to incubating sperm, as demonstrated by a peak shift to the right (i-iv), when compared to the carrier control. Under identical conditions, this enhancement was negated when ApoJ Ab (1:100) was added to the lipoproteins rather than PIS to block ApoJ, as demonstrated by the absence of a peak shift to the right (v-viii).

It is important to understand that lipid carriers, such as ApoJ/clusterin, that are effective in the invention, are preferably membrane free, and, as such it is certainly anticipated, and well within the means of those having skill in the art, that recombinant means can be used to promote the process of the invention.

Compositions comprising a substantially purified bioactive molecule and a lipid carrier for administration to animals, can be prepared by techniques known to those skilled in the art. For example, a purified preparation can be obtained following an individual technique or a series of preparative or biochemical techniques. The procedures can include, for example, but are not limited to, ammonium sulfate fractionation, gel filtration, ion exchange chromatography, affinity chromatography, density gradient centrifugation and electrophoresis. Recombinant proteins can be made by a variety of methods including but not limited to transformation, phage introduction, and non-bacterial transformation.

One method of preparation of a substantially purified bioactive molecule or lipid carrier of the invention is using recombinant means. Recombinant bioactive molecules, including GPI-linked proteins, and lipid carriers may be produced and purified by known techniques, such as those described in US Publication Nos. 2004/0268425 and 2007/0197466. The entirety of both references are herein incorporated by reference.

For example, one aspect of the invention pertains to vectors, containing the sequence encoding the desired protein of the invention, for example, a nucleic acid encoding a bioactive molecule, such as GPI-linked protein or a lipid carrier such as clusterin or derivatives thereof for its convenient cloning, amplification, and/or transcription. As used herein, the term “vector” refers to a nucleic acid molecule capable of transporting another nucleic acid to which it has been “operably linked.” One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the transcription of sequences to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), and artificial chromosomes, which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be transcribed. Within a recombinant expression vector, “operably-linked” is intended to mean that the nucleotide sequence of interest is linked to the regulatory sequence(s) in a manner that allows for transcription and/or expression of the nucleotide sequence (e.g., in an in vitro transcription/translation system or in a host cell when the vector is introduced into the host cell).

The term “regulatory sequence” is intended to include promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of transcription, and/or expression of protein desired, etc. The expression vectors of the invention can be introduced into host cells to thereby produce proteins or peptides, including fusion proteins or peptides, encoded by nucleic acids as described herein. The recombinant expression vectors of the invention can be designed for transcription and/or expression in prokaryotic or eukaryotic cells. For example, transcription and/or expression in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif (1990). Alternatively, the recombinant expression vector can be transcribed and/or translated in vitro, for example using T7 promoter regulatory sequences and T7 polymerase.

In another embodiment, the recombinant vector is capable of directing transcription of the sequence encoding the desired protein preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the alpha-fetoprotein promoter (Campes and Tilghman, 1989. Genes Dev. 3: 537-546).

In other aspects, the invention relates to a host cell comprising the sequence encoding the desired protein of the invention. In certain embodiments, the host cell comprises a vector, plasmid or artificial chromosome nucleic acid containing one or more transcription regulatory nucleic acid sequences operably linked with the sequence encoding the desired protein of the invention. The vector or plasmid nucleic acids can be, for example, suitable for eukaryotic or prokaryotic cloning, amplification, or transcription. In other embodiments, the invention comprises a plurality of aptameric GRO sequences linked contiguously as a single polynucleotide chain. In still other embodiments, the invention comprises a nucleic acid vector containing a plurality the sequences encoding the desired protein linked contiguously and operably linked with the nucleic acid sequence of the vector.

The term “host cell” includes a cell that might be used to carry a heterologous nucleic acid, or expresses a peptide or protein encoded by a heterologous nucleic acid. A host cell can contain genes that are not found within the native (non-recombinant) form of the cell, genes found in the native form of the cell where the genes are modified and re-introduced into the cell by artificial means, or a nucleic acid endogenous to the cell that has been artificially modified without removing the nucleic acid from the cell. A host cell may be eukaryotic or prokaryotic. For example, bacteria cells may be used to carry or clone nucleic acid sequences or express polypeptides. General growth conditions necessary for the culture of bacteria can be found in texts such as BERGEY'S MANUAL OF SYSTEMATIC BACTERIOLOGY, Vol. 1, N. R. Krieg, ed., Williams and Wilkins, Baltimore/London (1984). A “host cell” can also be one in which the endogenous genes or promoters or both have been modified to produce the sequence encoding the desired protein of the invention.

Protein purification can be preformed by any method known to one of skill in the art. These methods include extraction, precipitation and differential solubilization, ultracentrifugation and chromatographic methods such as size exclusion chromatography, separation based on charge or hydrophobicity, ion exchange chromatography, affinity chromatography, metal binding, and immunoaffinity chromatography. Purification may be preparative or analytical.

Extraction: Depending on the source, the protein is brought into solution by breaking the tissue or cells containing it by several known methods, such as repeated freezing and thawing, sonication, homogenization by high pressure or permeabilization by organic solvents. After this extraction process soluble proteins may be in the solvent, and can be separated from cell membranes, DNA etc. by centrifugation.

Precipitation and differential solubilization: In bulk protein purification, protein a re isolated by precipitation with ammonium sulfate. This is performed by adding increasing amounts of ammonium sulfate and collecting the different fractions of precipitate protein.

Ultracentrifugation: Centrifugation is a process that uses centrifugal force to separate mixtures of particles of varying masses or densities suspended in a liquid. When a vessel (typically a tube or bottle) containing a mixture of proteins or other particulate matter, such as bacterial cells, is rotated at high speeds, the angular momentum yields an outward force to each particle that is proportional to its mass. The tendency of a given particle to move through the liquid because of this force is offset by the resistance the liquid exerts on the particle. The net effect of spinning the sample in a centrifuge is that massive, small, and dense particles move outward faster than less massive particles or particles with more drag in the liquid. When suspensions of particles are spun in a centrifuge, a pellet may form at the bottom of the vessel that is enriched for the most massive particles with low drag in the liquid. The remaining, non-compacted particles still remaining mostly in the liquid are called the supernatant and can be removed from the vessel to separate the supernatant from the pellet. The rate of centrifugation is specified by the angular acceleration applied to the sample, typically measured in comparison to the g. If samples are centrifuged long enough, the particles in the vessel will reach equilibrium wherein the particles accumulate specifically at a point in the vessel where their buoyant density is balanced with centrifugal force. Such an “equilibrium” centrifugation can allow extensive purification of a given particle.

Chromatographic methods: A protein purification protocol may contain one or more chromatographic steps. The basic procedure in chromatography is to flow the solution containing the protein through a column packed with various materials. Different proteins interact differently with the column material, and can thus be separated by the time required to pass the column, or the conditions required to elute the protein from the column. Usually proteins are detected as they are coming off the column by their absorbance at 280 nm. Many different chromatographic methods exist, including size exclusion chromatography, separation based on charge or hydrophobicity, ion exchange chromatography, affinity chromatography, metal binding, and immunoaffinity chromatography

These compositions can be prepared to deliver an effective amount or dose of bioactive molecule and/or lipid carrier. An effective dose is an amount that is effective in the remodeling of sperm cells. An effective dose is also an amount that is effective in increasing the likelihood of fertilization.

In determining an effective amount or dose of bioactive molecule and/or lipid carrier, a number of factors are considered by the attending diagnostician, including, but not limited to: the species of the mammal; its size, age, and general health; the response of the individual patient or sperm; the particular bioactive molecule administered; the particular carrier administered, the mode of administration; the characteristics of the preparation administered; the dose regimen selected; the use of concomitant medication; and other relevant circumstances.

The composition of the invention can be administered in any form or mode which makes the bioactive molecule and carrier effective. Suitable modes of administration include oral, inhalation, nasal, buccal, topical, rectal, sublingual, transdermal, vaginal, otic, ophthalmic or parenteral administration. Parenteral administration may include intratracheal or inhalant aerosol administration, subcutaneous injection, intravenous injection, intraperitoneal injection, intramuscular injection, intrasternal injection, intrathecal injection, intraventricular and intracerebroventricular injection and infusion techniques. Transdermal and vaginal compositions are generally preferred. One skilled in the art of preparing formulations can readily select the proper form and mode of administration depending upon the relevant circumstances.

A bioactive molecule and carrier of the invention can be administered in the form of pharmaceutical compositions or medicaments which are made by combining a bioactive molecule and a carrier, with pharmaceutically acceptable carriers or excipients, the proportion and nature of which are determined by the chosen route of administration, and standard pharmaceutical practice. The term “pharmaceutically acceptable” refers to a molecular entity or composition that does not produce an allergic or similar unwanted reaction when administered to animals or humans.

The pharmaceutically acceptable carriers used in conjunction with the bioactive molecules and lipid carriers of the present invention vary according to the mode of administration. Solid carriers suitable for use in the composition of the invention include one or more substances which may also act as flavoring agents, lubricants, solubilizers, suspending agents, fillers, glidants, compression aides, binders, tablet-disintegrating agents or encapsulating materials. In powders, the carrier may be a finely divided solid forming an admixture. In tablets, the carrier may be mixed to provide the necessary compression properties in suitable proportions and compacted in the shape and size desired. Solid carriers suitable for use in the composition of the invention include calcium phosphate, magnesium stearate, talc, sugars, lactose, dextrin, starch, gelatin, cellulose, methyl cellulose, sodium carboxymethyl cellulose, polyvinylpyrrolidine, low melting waxes and ion exchange resins.

Liquid carriers suitable for preparing solutions, suspensions, and emulsions may be employed in the composition of the invention. The actives may be dissolved or suspended in a pharmaceutically acceptable liquid carrier such as water, an organic solvent, or a pharmaceutically acceptable oil or fat, or a mixture thereof. Said liquid composition may contain other suitable pharmaceutical additives such as solubilizers, emulsifiers, buffers, preservatives, sweeteners, flavoring agents, suspending agents, thickening agents, coloring agents, viscosity regulators, stabilizers, osmo-regulators, or the like.

The compositions or medicaments are prepared in a manner well known in the pharmaceutical art. The carrier or excipient may be a solid, semi-solid, or liquid material that can serve as a vehicle or medium for the active ingredient. Suitable carriers or excipients are well known in the art.

The concentration of bioactive molecule and carrier can vary widely as a function of the age, weight and state of health of the patient, the nature and level of need for sperm enhancement, as well as of the administration route. These concentration ranges can naturally be adjusted for each patient according to the results observed. The percentage of bioactive molecule in the composition or present in the medium or capacitation mediums may range from about 0.01% to about 99.9%. The percentage of lipid carrier in the composition or present in the medium or capacitation mediums may range from about 0.01% to about 99.9%.

### EXAMPLES

### Example 1

For ELF and ULF in vitro transfer of SPAM1 (and a related hyaluronidase, HYAL3) occurs efficiently from both the 120,000×g pellet (120P) vesicles and the soluble 120,000×g supernatant (120S) fractions, with the latter being greater.

Procedure

ELF was collected from the epididymides of sexually mature males as described, and centrifuged at 16,100×g to pellet cellular fragments and sperm. The supernatant was confirmed to be sperm-free after microscopic examination. ULF collected by flushing uteri from superovulated females, was also clarified via centrifugation. Ultracentrifugation (120,000×g for 2 hr at 4° C.) of the LFs was performed to separate the vesicular (120P) from the soluble (120S) fraction. Caudal sperm were collected as described. They were exposed to unfractionated ULF and ELF and their fractions at a concentration of ˜1 mg/ml protein for 2 hr at 37° C. and 32° C., respectively. After incubation, immunocytochemistry was performed and flow cytometric analysis used to quantify SPAM1 acquisition, as compared to sperm incubated in Bovine Serum Albumin (BSA) or Phosphate Buffered Saline (PBS) (which were negative controls). Similar experiments were performed to test for HYAL3, a related hyaluronidase, in ELF and all experiments were repeated twice.

Results and Interpretation

Sperm exposed to unfractionated LF acquired considerable amounts of SPAM1 compared to the control. Unexpectedly, sperm exposed to the 120S acquired more SPAM1 than those exposed to the 120P. This was also the case for HYAL3. The findings are similar to those for the intake of GPI-linked proteins by Chinese Hampster Ovary (CHO) cells and RBC incubated in seminal plasma and show that transfer from the soluble phase is more efficient than that from vesicles.

### Example 2

SPAM1 acquisition is targeted to the PM of sperm and not to that of RBC and the localization may pattern may depend on the fraction.

Introduction and Rationale

Since RBC share with sperm the ability for uptake of GPI-linked proteins, it was important to determine if SPAM1 could also be transferred to their PM. Thus experiments were performed using mouse RBC for uptake from unfractionated ULF under the identical conditions used for sperm binding. Additionally Spam1 null mice were utilized to investigate if localization of uptake on the PM was influenced by the specific fraction of ULF, 120S versus 120P.

Results and Interpretation

While sperm acquired SPAM1 as demonstrated by an increase in fluorescence intensity, there was no transfer for RBC. Because RBC are known to carry GPI-linked proteins, acquired from the plasma the lack of SPAM1 acquisition in RBC suggests that sperm may have specific lipid raft associated microdomains within the PM for SPAM1 binding or adsorption, or alternatively, that there may be specific sperm receptor(s) that mediate the binding. Immunocytochemical images localize SPAM1, acquired from ULF unfractionated and fractionated, to regions of the sperm PM directly overlying the acrosomal cap, and to the midpiece of the tail. However in a large number of cells, the distribution of SPAM1 was distributed throughout the midpiece of the tail. This distribution pattern suggests that initially there may be random insertion in the PM followed by migration of the protein to the localized areas over the acrosome.

### Example 3

Repeat ultracentrifugation of LFs with increasing force enrich for SPAM1 monomers which are the primary vehicles of transfer in the liquid phase.

Introduction and Rationale

Although for ELF the proportion of SPAM1 in the 120S and 120P fractions is 40:60, the 120S fraction appears to be more efficient in transferring SPAM1 and HYAL3 to the PM, as is the case for ULF. Membrane-free transfer of GPI-linked proteins has been documented from seminal plasma and filtered blood plasma, but to date this is the first demonstration of transfer for membrane-free GPI-linked proteins from the ELF and ULF. Since GPI anchors are highly hydrophobic, multiple GPI-linked molecules are expected to aggregate due to a more favorable level of entropy. Thus, monomers are likely to be in equilibrium with oligomers, with the concentration of the protein determining the proportion of each of these fractions. The equilibrium would shift towards aggregates when the critical micellar concentration is present, and towards monomers when the amount of protein falls below this level. Thus it is important to investigate the physical nature of the soluble-SPAM1 in the liquid phase.

Procedure

ELF was subjected to ultracentrifugation at 120,000×g for 2 hr. The resulting supernatant was centrifuged at 150,000×g for 4 hr. This process was repeated at speeds of 190,000 (8 hr) and 230,000×g (16-24 hr). All pellets were resuspended in the initial volume of 5 ml to determine the relative concentration and form of SPAM1 in each fraction. Equal volumes of each sample were subjected to native PAGE and Western blot analysis.

Results and Interpretation

Both monomeric and high MW forms of SPAM1 were detected for each fraction, however the proportion of each varied among fractions. Monomeric SPAM1 (67 kDa) was relatively enhanced with sequential repeat utracentrifugation and was most abundant in the 230S fraction. This indicates that either removal of SPAM1 drops its concentration below that of the critical micellar concentration necessary for aggregation of monomers (high MW smears), or that oligomeric SPAM1 can be pelleted via ultracentrifugation. These results also demonstrate that LF 230S is monomer-rich.

### Example 4

Characterization of the Monomer-rich fraction (230S) and its ability to Transfer SPAM1 in LFs Rationale.

Since GPI anchors are highly hydrophobic, the transport of membrane-free GPI-linked molecules within an aqueous solution is highly unlikely without an amphipathic carrier. It is proposed, as depicted in FIG. 2, that lipoproteins which are abundant in the LFs could function as carriers of these proteins since they are well-known acceptors for cholesterol. Thus, the affinity of the monomer-rich 230S fraction will be determined, among the others, for lipoproteins. Then it will be investigated if it can transfer SPAM1, and its efficiency in doing so relative to the 230P fraction. Finally, it will be determined how exogenous lipoproteins might affect the ability of the 230S fraction to transfer SPAM1.

Procedure

Fractions separated in Example 3 were subjected to native gel electrophoresis to detect their association with lipoproteins, using a rat anti-HDL antibody (prepared by Prof. David Usher in our Department), with a broad specificity for HDL, ApoA-1, and ApoE for Western analysis.

ELF 120S from mature males was subjected to ultracentrifugation at 230,000×g for 2 hr to pellet all membranous vesicles. Caudal sperm were incubated in ELF 230S, ELF 230P or BSA under aforementioned conditions. After incubation, sperm were immunostained for SPAM1 and analyzed for SPAM1 uptake via flow cytometry.

Lipoproteins were isolated by density ultra-centrifugation from rat serum. ELF 230S samples were treated with increasing concentrations of rat lipoproteins before incubation with caudal sperm; sperm incubated in NaCl carrier and BSA were used as a control. Sperm were analyzed for SPAM1 acquisition via flow cytometry.

Results and Interpretation

Native PAGE gel electrophoresis showed ELF supernatants to be more highly associated with lipoproteins than were the pellets. Western analysis of the various fractions of ELF showed no and low association with the pellets at 120P (vesicles) and 230P (aggregates), respectively; but high association with both supernatants, with the 230S being greater than the 120S. Thus there is a direct relationship between the proportion of monomers and the level of associated lipoproteins.

Caudal sperm incubated in ELF 230S acquired demonstrable levels of SPAM1 when compared to those incubated in BSA as determined by an increase in fluorescence intensity, yet those incubated in 230P demonstrated comparatively negligible SPAM1 uptake. This indicates that the primary form of SPAM1 that is transferred to the sperm surface from LF 120S resides in 230S.

Finally, when various amounts of exogenous rat lipoproteins were added to the 230S ELF supernatant SPAM1 transfer to the sperm PM was inhibited in a concentration-dependent manner, implicating the involvement of lipoproteins in transfer. Alternatively, lipoproteins could sequester SPAM1 making it inaccessible for transfer, or saturate monomeric SPAM1 uptake sites on sperm.

### Example 5

ApoJ Antibodies inhibit SPAM1 transfer from the 230S fraction in ELF/ULF and co-immunoprecipitation reveals an association of ApoJ and SPAM1.

Introduction

With the results of the previous experiment implicating the involvement of lipoproteins in SPAM1 uptake from the 230S, it was important to block one of our candidates to determine its impact on transfer. Rat ApoJ antibody was provided to us from the laboratory of Dr. Michael Griswold, Washington State University for this purpose.

Procedure

The ApoJ antibody (Ab) is polyclonal and was generated in rabbit. Since SPAM1 antibody is also a rabbit polyclonal antibody, it was important to remove the AopJ antibody (Ab) from the sperm after incubation in the 230S before immunodetection of SPAM1. Several dissociating agents at extremes of salinity and pH were tested for their ability to remove ApoJ, with 1 M KCl (pH 7.2) giving the best results. With this reagent, virtually all of the ApoJ antibody could be stripped from the sperm prior to immunodetection of SPAM1. Thus 1 M KCl was used for all the experiments prior to quantitation of SPAM1 uptake in the presence of ApoJ.

WT ELF or ULF was subjected to centrifugation for 3 hr at 230,000×g. Caudal sperm (from the ELF donors) were washed, and incubated in PBS, or LF 230S+preimmune serum (PIS) or 230S+ApoJ Ab (both 1:1000) for 2 hr. After uptake, sperm were washed twice in PBS, and subjected to 1M KCl (pH 7.2) for 15 min at RT to remove ApoJ Ab bound to the sperm surface. Sperm were then washed 3 times in PBS, and processed for SPAM1 detection with our primary SPAM1 Ab (1:320) and FITC-conjugated secondary Ab (1:400) followed by flow cytometric analysis.

To determine an association of SPAM1 and ApoJ, immunoprecipitation was performed on the 230S fraction. ELF/ULF 230S was treated with PIS or SPAM1 Ab (1:1000), overnight, (4° C.). Samples (1 ml) were incubated with 125 μl Seize X Protein A beads (Pierce) overnight at 37° C. Beads were washed 3× in 1× PBS and treated with 100 mM DTT in sample loading dye and heated to 60° C. for 5 min. to extract immunoprecipitated proteins. Samples were probed for the presence of SPAM1 and ApoJ via Western analysis.

Results and Interpretation

Data show that there was a remarkable degree of inhibition of SPAM1 uptake from both ELF and ULF. This strongly implicates ApoJ in the transfer of SPAM1 from both the epididymal and uterine secretions. Importantly, it also demonstrates an association between SPAM1 and ApoJ in the LFs. Western blot analysis indicates that these two proteins can be co-immunoprecipitated. This finding reveals an association of the proteins and suggests that they might be interacting. Such an interaction could identify a role for ApoJ in the transfer of SPAM1 and other GPI-linked proteins from ELF and ULF to the sperm PM.

Although the invention is illustrated and described herein with reference to specific embodiments, the invention is not intended to be limited to the details shown. Rather, various modifications may be made in the details within the scope and range of equivalents of the claims and without departing from the invention.

### Example 6

Lipoprotein Isolation

Rat serum lipoprotein was isolated by density ultracentrifugation. Rat blood was subjected to centrifugation at 2,000×g for 20 min to pellet red blood cells (RBCs). The density of the resulting rat serum was increased to 1.21 g/mL by adding 1.41 g. sodium bromide (NaBr) to a final volume of 5 mL (adjusted with water to a final weight of 6.05 g) and ultracentrifuged at 230,000×g for 48 hr. The protein concentration of the resulting supernatant was determined by a biocinchoninic acid assay (BCA kit, Pierce). It was shown to contain lipoproteins via dot blot analysis for high density lipoprotein (HDL) and ApoJ. For clinical trials, purified human ApoJ that is commercially available (Millipore) alleviates the need to use the crude lipoprotein extract from rats.

### Example 7

In Vitro fertilization

In vitro fertilization will be performed by retrieving egg(s) from the ovary of a female. The eggs will be retrieved using (from) known techniques, such as a transvaginal technique involving an ultrasound-guided needle piercing the vaginal wall to reach the ovaries. The needle follicles will be aspirated, and the follicular fluid handed to the IVF laboratory to identify ova. The retrieval procedure will take about 20 minutes and will usually be done under conscious sedation or general anesthesia. Sperm will be collected from the male using known techniques. An aliquot of about 10⁶ sperm will be combined with about 100 ng/mL to about 100 ug/mL of human ApoJ solubilized recombinant SPAM1 protein extract from the surface of CHO cells and about 0.1 to about 100 ug/mL, preferably about 1.0 to about 40 ug/mL and most preferably about 5 to about 20 ug/mL of lipid carrier in conventional in vitro fertilization apparatus. This combination will be performed both in the presence of the egg and outside the presence of the egg. The concentration, time and other conditions used will be optimized to achieve maximum transfer of SPAM1 to the sperm prior to interaction with the egg. When performed outside the presence of the egg, the egg will be introduced to the combined mixture. The combination of egg, sperm, ApoJ and SPAM1 will be maintained under normal in vitro fertilization conditions until fertilization of the egg is achieved. After fertilization, the fertilized egg will be cultured under normal culturing conditions until the fertilized egg produces an embryo. Thereafter, the embryo will be transferred using known transfer techniques into the uterus of a female.

### Example 8

IUI

IUI will be performed by collecting sperm from a male using known techniques. An aliquot of about 10⁶ sperm will be combined with about 100 ng/mL to about 100 ug/mL of human ApoJ solubilized recombinant SPAM1 protein extract from the surface of CHO cells and about 0.1 to about 100 ug/mL, preferably about 1.0 to about 40 ug/mL and most preferably about 5 to about 20 ug/mL of lipid carrier in conventional IUI apparatus. The combination of sperm, ApoJ and SPAM1 will be maintained under normal IUI conditions. The concentration, time and other conditions used will be optimized to achieve maximum transfer of SPAM1 to the sperm prior to interaction with the egg. Thereafter, the SPAM1 enhanced sperm will be transferred using known transfer techniques into the uterus of a female for fertilization of an egg.

### Example 9

Introduction: Fertilization is dependent on a series of required steps that begin with the penetration of the cumulus matrix by sperm, via their neutral hyaulronidase activity. Since SPAM1 plays a role in several of these steps, it is important to determine if its transfer from the soluble fraction of LFs to the sperm PM increases sperm maturation and fertilizing ability. (See FIG. 6) Functional studies to determine the impact of SPAM1 transfer from the more efficient membrane-free fraction is needed. As a preliminary test, the ability of SPAM1 null sperm to penetrate the cumulus after SPAM1 transfer from unfractionated ELF was assessed. Also assessed was whether murine SPAM1 is involved in HA-enhanced progesterone-induced acrosome reaction, a known functional test for human sperm, in order to determine if the test could be used in AIM III.

Procedure: Working with Dr. Ron Feinberg of the Reproductive Associates of Delaware (Newark, Del.) semen samples were obtained from men undergoing IVF or ISCI, for this purpose. Liquefied semen samples were obtained from the clinic. Sperm were then washed in PBS and proteins extracted in solubilization buffer to determine the level of hyaluronidase activity, using hyaluronic acid (HA) substrate gel electrophoresis. HA Substrate Gel Electrophoresis (HASGE) SPAM1 hyaluronidase activity in sperm protein extracts was measured. Briefly, HA from bovine vitreous humor was added to a 10% SDS-polyacrylamide gel (final concentration 0.3 μg/mL). Gels were loaded with 20 μg of non-reduced proteins and run at 15 mA. After completion, they were incubated in 3% Triton X-100 in PBS for 2 hr at RT, then at 37C for 36 hr in 100 mM sodium acetate (pH 7.0). To visualize digestion of HA, gels were stained with 0.5% alcian blue in 3% acetic acid for 2 hr, and destained in 7% acetic acid until digestion was visible. Gels were counterstained with Coomassie Brilliant Blue G-250 and destained with methanol-acetic acid.

FIG. 7 shows the results from 6 men studied consecutively between June and August in 2006. It is unknown which males are from couples with male- or female-factor infertility. It is evident that there is a large variation in the level of hyaluronidase activity seen in this small sample: 1/6 or 16.6% has no activity and 3/6 had drastically reduced activity. Whether or not these men are representative of the population is also unknown, but the data clearly shows substantial variation. This work suggests that there will be a proportion of males who might benefit from delivery of SPAM1 in vitro during capacitation for IVF or prior to intrauterine insemination, to improve sperm fertilizing ability. Although low hyaluronidase activity might not be equivalent to low SPAM1 protein level, it is likely that there will be some individuals who will have sperm with the capacity to acquire exogenous SPAM1.

The entire disclosures of all applications, patents and publications, cited above and below are hereby incorporated by reference.

What is claimed:

1. A composition comprising a bioactive molecule and a lipid carrier, wherein at least one of said bioactive molecule or said lipid carrier is substantially purified and wherein said bioactive molecule is selected from the group consisting of GPI-linked proteins, enzymes, adhesion molecules, immune proteins, antigens and glycoproteins.

2. The composition of claim 1 wherein the bioactive molecule is selected from the group consisting of sperm adhesion molecule 1 (SPAM1) and P34H.

3. The composition of claim 1 where the bioactive molecule is membrane free.

4. The composition of claim 1 wherein the lipid carrier is selected from the group consisting of Clusterin, ApoJ, Clusterin/ApoJ, ApoA-1, SGP2, TRPM, gp80 and SP-40.

5. A method of enhancing fertilization comprising administering the composition of claim 1 to an animal whereby the bioactive molecule is transferred from the composition to the surface of a sperm cell in the animal.

6. The method of claim 5 wherein the bioactive molecule selected from the group consisting of GPI-linked proteins, enzymes, adhesion molecules, immune proteins, antigens and glycoproteins.

7. The method of claim 5 wherein the bioactive molecule is selected from the group consisting of sperm adhesion molecule 1 (SPAM1) and P34H.

8. The method of claim 5 wherein the bioactive molecule is membrane free.

9. The method of claim 5 wherein the lipid carrier is selected from the group consisting of Clusterin, ApoJ, Clusterin/ApoJ, ApoA-1, SGP2, TRPM, gp80 and SP-40.

10. A method for enhancing sperm maturation and function, the method comprising, in an in vitro environment:
(a) isolating sperm from a male candidate, and
(b) adding the sperm in vitro to a medium wherein the medium is supplemented with a molecule selected from the group consisting of a lipid carrier a bioactive molecule, or a combination of both.

11. The method of claim 10 further comprising delivering said sperm to the uterine tract.

12. The method of claim 10 wherein the bioactive molecule is selected from the group consisting of GPI-linked proteins, enzymes, adhesion molecules, immune proteins, antigens and glycoproteins.

13. The method of claim 10 wherein the bioactive molecule is selected from the group consisting of sperm adhesion molecule 1 (SPAM1) and P34H.

14. The method of claim 10 wherein the bioactive molecule is membrane free.

15. The method of claim 10 wherein the lipid carrier is selected from the group consisting of Clusterin, ApoJ, Clusterin/ApoJ, ApoA-1, SGP2, TRPM, gp80 and SP-40.

16. The method of claim 10 wherein the medium comprises capacitation medium.

17. The method of claim 16 further comprising adding at least one egg to the medium and incubating until said egg is fertilized.

18. The method of claim 17 further comprising cultivating said fertilized egg into an embryo.

19. The method of claim 18 further comprising delivering said embryo to a uterine tract.

20. A method for removing a GPI-linked protein from a cell, the method comprising the step of adding at least 40 ug/mL of a lipid carrier to said cell.

21. The method of 20 wherein the GPI-linked proteins is selected from the group consisting of sperm adhesion molecule 1 (SPAM1) and P34H.

22. The method of 20 wherein the lipid carrier is selected from the group consisting of Clusterin, ApoJ, Clusterin/ApoJ, ApoA-1, SGP2, TRPM, gp80 and SP-40.

23. The method of 20 wherein the concentration of lipid carrier is about 40 to about 2000 ug/mL.
